(19) United States
(12) Patent Application Publication
(10) Pub. No.: US 2025/0127970 A1
(43) Pub. Date: Apr. 24, 2025

(54) GENERATING MEDICAL FLUID FOR RENAL REPLACEMENT THERAPY

(71) Applicant: GAMBRO LUNDIA AB, Lund (SE)

(72) Inventors: Dominique POUCHOULIN, Tramoyes (FR); Michael PETTERSSON, Malmö (SE); Per-Olof BORGQVIST, Lund (SE); Jonas FORS, Malmö (SE); Olof JANSSON, Vellinge (SE)

(21) Appl. No.: 18/682,793

(22) PCT Filed: Aug. 8, 2022
(86) PCT No.: PCT/EP2022/072179

§ 371 Date: Feb. 9, 2024

(30) Foreign Application Priority Data
Aug. 9, 2021 (SE) 2150990-6

(51) Int. Cl. A61M 1/16

(52) U.S. Cl. CPC A61M 1/1668; A61M 2205/10; A61M 2205/12; A61M 2205/3327; A61M 2205/3334; A61M 2205/3365

(57) ABSTRACT

A medical fluid for use in treatment of blood by renal replacement therapy, RRT, is generated in a system comprising pumps for pumping fluids from containers into a fluid channel for mixing therein. The pumps are controlled based on output signals from scales, on which the containers are arranged, to achieve a given mixing ratio between the fluids. The system may be configured by arranging a disposable arrangement on a machine comprising the pumps and the scales. The disposable arrangement may define the fluid channel and the containers, and the machine may be an RRT apparatus. The system may be operated to generate the medical fluid on-line for the RRT apparatus.

### TECHNICAL FIELD

The present disclosure relates to the field of renal replacement therapy and in particular to generation of a medical fluid for use in such therapy.

### BACKGROUND ART

Renal replacement therapy (RRT) is a therapy that replaces the normal blood-filtering function of the kidneys. It is used when the kidneys are not working well, which is known as kidney failure and includes acute kidney injury and chronic kidney disease. RRT involves removal of solutes from the blood of a patient suffering from kidney failure, for example by dialysis (hemodialysis, HD, or peritoneal dialysis, PD), hemofiltration, or hemodiafiltration. Depending on modality, RRT may be performed manually or by use of a machine.

In RRT, one or more medical fluids of specific composition are used for treatment of blood. Such medical fluids include so-called dialysis fluid and replacement fluid. Over time, RRT consumes large quantities of medical fluid.

In some modalities of RRT, pre-made medical fluid is delivered in prefilled bags to the point of care, for example an intensive care unit or the home of the patient. The use of large quantities of medical fluid has significant environmental impact through transportation. In an intensive care unit, the administration and handling of prefilled bags at the point of care is taxing on the staff, takes time and diverts the attention of the staff from other tasks.

For example, conventional PD is performed by use of prefilled bags. In HD, different types of machines are used for treatment of patients with acute kidney injury (AKI) and patients with chronic kidney disease (CKD). HD machines for treatment of patients with AKI are generally configured to use prefilled bags of medical fluid, whereas HD machines for treatment of patients with CKD generally have integrated capability to generate medical fluid on-demand by mixing one or more concentrates with water, so-called on-line fluid generation. Recently, PD machines with integrated capability of on-line fluid generation have also been proposed.

A machine for RRT with integrated fluid generation capability is relatively complex and costly in comparison to a machine for RRT that is configured to use prefilled bags of medical fluid.

### SUMMARY

It is an objective to at least partly overcome one or more limitations of the prior art.

A further objective is to reduce the complexity of a machine for generating medical fluid for use in treatment of blood by RRT.

Another objective is to reduce the cost of generating a medical fluid by use of a machine.

One or more of these objectives, as well as further objectives that may appear from the description below, are at least partly achieved by a method of generating a medical fluid, a computer-readable medium, a system for generating a medical fluid, and a disposable arrangement, embodiments thereof being defined by the dependent claims.

A first aspect is a method of generating a medical fluid for use in treatment of blood by renal replacement therapy. The method comprises: operating a first pump to pump a first fluid from a first container arranged on a first scale, through a first fluid channel, the first fluid being a component of the medical fluid; operating a second pump to pump a second fluid from a second container arranged on a second scale, through a second fluid channel into the first fluid channel at a first junction in the first fluid channel, to admix the second fluid within the first fluid channel, the second fluid being a component of the medical fluid; and controlling the first and second pumps, based on first and second output signals from the first and second scales, to achieve a first proportion between a first flow rate of the first fluid into the junction and a second flow rate of the second fluid into the junction.

The first aspect controls the generation of the medical fluid based on the first and second output signals, which represent measurements by the first and second scales and thus the consumption of the first and second fluids. Thereby, the first and second output signals indicate changes in mass or weight over time and thus contain information about the mass flow rates of the first and second fluids during the generation of the medical fluid. The first aspect thereby provides a simple and well-controlled way of generating the medical fluid, by controlling the mixing ratio of the first and second fluids based on flow rates given by the output signals of the scales (gravimetric flow measurement). Further, according to the first aspect, the second fluid is admixed within the first fluid channel itself. The first aspect is based on the insight that sufficient mixing may be achieved in the first fluid channel, without the need for a conventional mixing tank or the like. The admixing of the second fluid inside the first fluid channel allows the medical fluid to be generated and supplied on-demand to a downstream apparatus for RRT, if desired. It also allows for reductions in size, structural complexity and cost of the system that generates the medical fluid.

A second aspect is a computer-readable medium comprising computer instructions which, when executed by a processor, cause the processor to perform the method of the first aspect or any of its embodiments.

A third aspect is a system for generating a medical fluid for use in treatment of blood by renal replacement therapy. The system comprises: a first scale; a first container arranged on the first scale; a first fluid channel arranged to receive a first fluid from the first container; a first pump arranged to pump fluid through the first fluid channel; a second scale; a second container arranged on the second scale and connected, by a second fluid channel, to the first fluid channel at a junction; and a second pump arranged to pump a second fluid from the second container through the second fluid channel into the first fluid channel to admix the second fluid within the first fluid channel.

The second and third aspects share technical advantages with the first aspect.

A fourth aspect is a disposable arrangement for mounting to an apparatus. The disposable arrangement comprises: a first container configured for mounting on a first scale of the apparatus; a first fluid channel arranged to receive a first fluid from the first container; and a second fluid channel connected to a junction on the first fluid channel; wherein the first fluid channel defines a first engagement portion for engagement with a first pump of the apparatus upstream of the junction, and wherein the second fluid channel defines a second engagement portion for engagement with a second pump of the apparatus for pumping a second fluid through the second fluid channel into the first fluid channel to admix the second fluid within the first fluid channel. The first and second fluids are components of a medical fluid for use in treatment of blood by renal replacement therapy, and the disposable arrangement is operable, when mounted on the apparatus, to generate the medical fluid in the first fluid channel.

The disposable arrangement of the fourth aspect provides a simple way of enabling an apparatus to be configured for generation of medical fluid. Any existing apparatus that comprises first and second scales and first and second pumps may be combined with the disposable arrangement to provide the new functionality of generating the medical fluid. For example, scales are common on RRT machines which are configured for so-called CRRT (Continuous Renal Replacement Therapy) and used for treatment of patients with AKI. It is realized that the fourth aspect provides a simple and cost-effective technique of generating medical fluid and obviates the need for an integrated fluid generation unit in the apparatus. The medical fluid may be generated on-line, which implies that the medical fluid is provided to an on-going RRT, which consumes the medical fluid at the rate it is generated. The on-going RRT may be performed by the apparatus itself, or by another apparatus for RRT. Alternatively, the medical fluid may be generated for storage in one or more containers, for subsequent distribution for use in RRT. The use of a disposable arrangement also mitigates or obviates the need for periodic disinfection of the apparatus, which is a necessity in any apparatus that has an integrated (permanent) unit for generation of the medical fluid.

Still other objectives, aspects and advantages, as well as features and embodiments, may appear from the following detailed description, from the attached claims as well as from the drawings.

### BRIEF DESCRIPTION OF THE DRAWINGS

FIG. 1 is a schematic diagram of an example system for generating a dialysis fluid.

FIG. 2A is a flow chart of an example method of generating a medical fluid for use in renal replacement therapy (RRT), and FIG. 2B is a flow chart of an example preparation process for RRT.

FIG. 3A is a front view of an example apparatus for RRT, FIG. 3B is a plan view of an example disposable arrangement for installation on the apparatus in FIG. 3A, and FIG. 3C is a plan view of an example mixing infusion device in the system of FIG. 1.

FIG. 4 is a flow chart of an example method of operating the system in FIG. 1.

FIGS. 5A-5B are schematic diagrams of variants of the system in FIG. 1.

FIG. 6 is a flow chart of an example verification procedure.

FIGS. 7A-7B are example graphs of pumping speed as a function of time during a verification procedure.

FIG. 8 is a schematic diagram of an example extracorporeal blood circuit for RRT.

FIG. 9 depicts a variant of the system in FIG. 1.

FIG. 10 depicts a further variant of the system in FIG. 1.

FIG. 11 is a flow chart of an example procedure performed by the system of FIG. 10.

### DETAILED DESCRIPTION OF EXAMPLE EMBODIMENTS

Embodiments will now be described more fully hereinafter with reference to the accompanying drawings, in which some, but not all, embodiments are shown. Indeed, the subject of the present disclosure may be embodied in many different forms and should not be construed as limited to the embodiments set forth herein; rather, these embodiments are provided so that this disclosure may satisfy applicable legal requirements. Like numbers refer to like elements throughout.

Also, it will be understood that, where possible, any of the advantages, features, functions, devices, and/or operational aspects of any of the embodiments described and/or contemplated herein may be included in any of the other embodiments described and/or contemplated herein, and/or vice versa. In addition, where possible, any terms expressed in the singular form herein are meant to also include the plural form and/or vice versa, unless explicitly stated otherwise. As used herein, “at least one” shall mean “one or more” and these phrases are intended to be interchangeable. Accordingly, the terms “a” and/or “an” shall mean “at least one” or “one or more,” even though the phrase “one or more” or “at least one” is also used herein. As used herein, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments.

It will furthermore be understood that, although the terms first, second, etc. May be used herein to describe various elements, these elements should not be limited by these terms. These terms are only used to distinguish one element from another. For example, a first element could be termed a second element, and, similarly, a second element could be termed a first element, without departing the scope of the present disclosure. As used herein, the terms “multiple”, “plural” and “plurality” are intended to imply provision of two or more elements. The term “and/or” includes any and all 20 combinations of one or more of the associated listed elements.

As used herein, “HD machine” refers to any machine that is dedicated to treatment of patients with AKI, known as “acute dialysis” in the art, and/or to treatment of patients with CKD, known as “chronic dialysis” in the art. Some embodiments are particularly suited for HD machines for acute dialysis since such HD machines generally comprise a set of scales.

As used herein, “medical fluid” refers to any fluid that may be supplied for use in blood treatment by renal replacement therapy (RRT), including dialysis fluid, replacement fluid (also known as substitution fluid) or any other infusion fluid. It may be noted that such a medical fluid is supplied for use in treatment of blood and is thus distinct from the blood as such. As used herein, RRT includes without limitation hemodialysis (HD), hemofiltration (HF), hemodiafiltration (HDF), peritoneal dialysis (PD), etc. The following description is applicable to any medical fluid that may be used for blood treatment by any form of RRT.

Well-known functions or structures may not be described in detail for brevity and/or clarity. Unless otherwise defined, all terms (including technical and scientific terms) used herein have the same meaning as commonly understood by one of ordinary skill in the art to which this disclosure belongs.

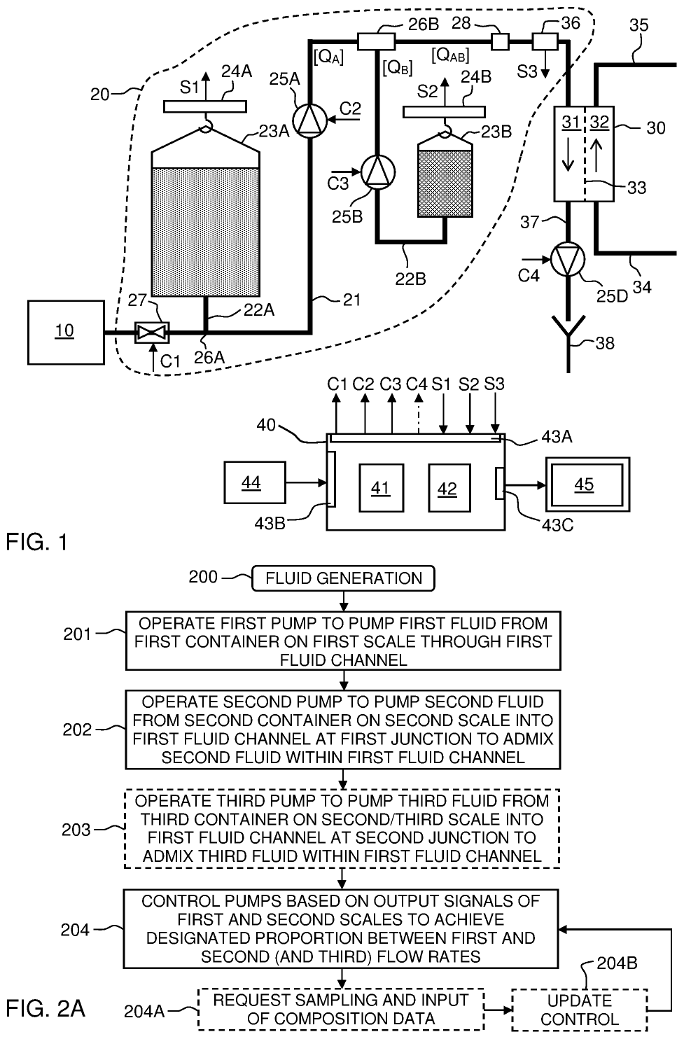

FIG. 1 is a schematic view of an example system 20 for generation of a medical fluid. The system 20 generates the medical fluid by mixing a first fluid with a second fluid. The first and second fluids are thus components of the medical fluid. The following description presumes that the first fluid is water and the second fluid is a liquid concentrate, and that the medical fluid is a dialysis fluid for use in HD. The system 20 is arranged to receive the water from a source 10, which is configured to supply water of required quality, for example in terms of contaminants, bacterial cell count, and endotoxins. The water source 10 may be a centralized system or a local stand-alone device connected to a tap water outlet.

The system 20 comprises a first fluid channel 21 (“main channel”) which extends from the source 10 to a blood filter (“dialyzer”) 30. A first container 23A is in fluid communication with the main channel 21 through a first connecting fluid channel 22A, which joins the main channel 21 at a junction 26A. A control valve 27 is arranged on the main channel 21 between the source 10 and the first connecting fluid channel 22A to control the admission of water into the system 20. The container 23A is arranged on a scale 24A, which is configured to provide a measurement signal or output signal S1 indicative of the weight of the container 23A. Although containers are shown herein as hung onto a hook-like member of the scale, they may alternatively be placed to rest on the scale. The system 20 further comprises a sub-system for feeding the concentrate into the main channel 21. The sub-system includes a second container 23B that contains the concentrate. The container 23B is arranged on a scale 24B, which is configured to provide a measurement signal or output signal S2 indicative of the weight of the container 23B. The container 23B is in fluid communication with the main channel 21 through a second connecting fluid channel 22B. The fluid channel 22B is connected to the main channel 21 at a junction 26B.

The system 20 further comprises two fluid pumps. A first fluid pump 25A is arranged in or on the main channel 21, intermediate junctions 26A, 26B, to pump water towards junction 26B along the main channel 21. A second fluid pump 25B is arranged to pump concentrate from the container 22B towards junction 26B, and thus into the main channel 21. In the following, the flow rates of water and concentrate into the junction 26B are denoted Q_{A} and Q_{B}, respectively, as indicated within brackets in FIG. 1. The flow rate of the resulting dialysis fluid is denoted Q_{AB}. The fluid flows of water and concentrate meet at the junction 26B and are mixed within the main channel 21 at and downstream of the junction 26B. Although not shown in FIG. 1, the system 20 may include one or more devices configured to promote the mixing, for example inside the junction 26B or downstream of the junction 26B in the main channel 21. In some embodiments, the junction 26B is a 3-way connector. Further details will be given below with reference to FIG. 3C.

In a variant, included in FIG. 10 (described below), the main channel 21 extends from the container 23A, which is in fluid communication with the source 10 through a separate fluid channel 29, and the control valve 27 is arranged on the separate fluid channel 29 to control the admission of water into the system 20.

In another variant, also included in FIG. 10, the first fluid pump 25A is instead arranged in or on the main channel 21 downstream of the junction 26B. Thereby, the pump 25A defines Q_{AB}, and also indirectly defines Q_{A} as Q_{B} is defined by pump 25B.

In the example of FIG. 1, the system 20 further includes a sampling port 28 downstream of the junction 26B. The sampling port 28 may be of any conventional configuration to provide access to the main channel 21 for extraction of a sample of the fluid therein.

As shown in FIG. 1, a sensor 36 may be arranged in the main channel 21 to measure the conductivity of the passing fluid or the concentration of one of more substances in the passing fluid. As shown, the sensor 36 provides a measurement signal or output signal S3.

In a further variant, not shown in FIG. 1, the system 20 further comprises one or more sterilizing grade filters, for example in the main channel 21 downstream of the junction 26B. The filter(s) may be configured to ensure that the medical fluid meets standards for ultrapure dialysis fluid or standards for replacement fluid in terms of viable bacteria (sterility) and endotoxins. Such filters are well-known in the art.

In FIG. 1, the system 20 is included in an arrangement for HD treatment and configured for on-line generation of dialysis fluid. On-line generation, as used herein, implies that the generation rate of the medical fluid matches the consumption rate of the medical fluid during RRT. The arrangement for HD treatment in FIG. 1 comprises a dialyzer 30, which defines first and second compartments 31, 32 that are separated by a semi-permeable membrane 33, as is well-known in the art. The main channel 21 is connected to the first compartment 31 to allow dialysis fluid to flow through the first compartment 31, as indicated by an arrow, into an effluent channel 37 which opens into a drain 38, as shown, or a container for collecting spent dialysis fluid (“effluent”). A further pump 25D (“effluent pump”) is arranged in or on the effluent channel 37 to control the flow rate of effluent from the dialyzer 30. As also indicated in FIG. 1, first and second blood lines 34, 35 are connected to the second compartment to allow blood from a patient to be pumped through the second compartment 32, as indicated by an arrow. Thereby, blood is treated in the dialyzer 30. The principle of hemodialysis is well-known to the skilled person and will not be further explained herein.

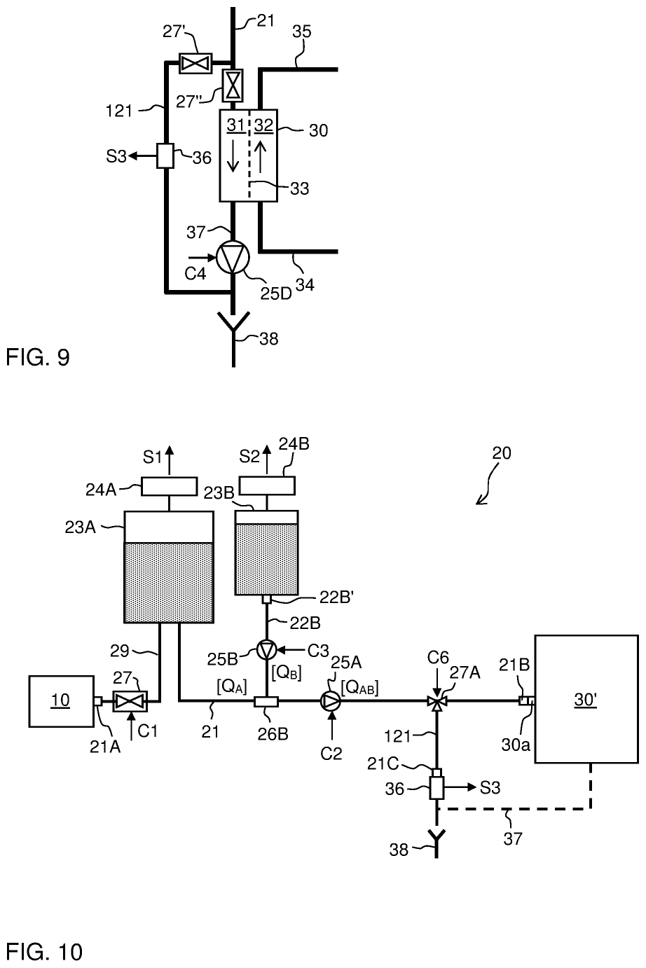

In a variant of the system 20, shown in FIG. 9, a bypass channel 121 is added in parallel with the first compartment 31 of the dialyzer 30. The bypass channel 121 establishes an additional fluid path between the main channel 21 and the drain 38. A valve arrangement 27′, 27″ is operable to selectively direct the flow in the main channel 21 into the bypass channel 121 instead of into the dialyzer 30. It is conceivable that the sensor 36, as shown, is arranged in the bypass channel 121. The flow of fluid is driven into and through the bypass channel 121 by the pumps 25A, 25B in FIG. 1.

In some embodiments, the system 20 is a permanent unit within an apparatus for RRT. In such a permanent unit, the fluid channels 21, 22A, 22B may be defined by tubing or configured as passageways in a solid block, and the fluid pumps 25A, 25B and the control valve 27 may be of any type. It is realized that the container 23B may be connected to a source of concentrate for refilling as required. Alternatively, the container 23B may be disconnected and replaced with a filled container when empty. It is understood that such a permanent unit needs to be connected to equipment for cleaning and disinfection of the fluid channels and any other component that encounters fluid.

In some embodiments, the system 20 is a permanent unit of a separate fluid generation apparatus which is arranged to supply medical fluid to an apparatus for RRT.

In some embodiments, which are described in further detail below with reference to FIGS. 3A-3B, the system 20 comprises a disposable arrangement that defines the fluid channels 21, 22A, 22B and the containers 23A, 23B and is arranged in engagement with an apparatus or machine that comprises other components of the system 20, such as scales, pumps, valves, etc. The machine may be an HD machine, also denoted “monitor” in the following, or a fluid generation apparatus which is separate from the HD machine. In the disposable arrangement, the fluid channels 21, 22A, 22B may be defined by tubing, and the containers 23A, 23B may be defined by flexible bags or rigid containers. In some embodiments, the disposable arrangement is made of plastic material. The fluid pumps 25A, 25B may be peristaltic pumps which engage the outside of the tubing to generate a moving compression of the tubing to force fluid to move along the tubing. Conventionally, to enable the use of a peristaltic pump, the tubing is provided with a dedicated engagement portion, also known as a pump segment, which is configured to be engaged by the compression element(s) of the peristaltic pump. Similarly, the valve 27 may be a clamp, pinch valve, or the like which engages the outside of the tubing to control the flow through the tubing, for example by selectively squeezing the tubing to block passage of fluid.

As shown in FIG. 1, a control device 40 is provided to control the operation of the system 20. If the system 20 is operated by an HD machine, the control device 40 may be a controller of the HD machine or a separate controller. In the illustrated example, the control device 40 is configured to generate a control signal C1 for the valve 27, and control signals C2, C3 for the pumps 25A, 25B in accordance with a control program comprising computer instructions. The control program is also configured to operate based on measurement signals S1-S3 received by the control device 40 from scales 24A, 24B and sensor 36 (if present). The control device 40 comprises a processor 41 and computer memory 42. The control program is stored in the memory 42 and executed by the processor 41. The control program may be supplied to the control device 40 on a computer-readable medium, which may be a tangible (non-transitory) product (e.g., magnetic medium, optical disk, read-only memory, flash memory, etc.) or a propagating signal. In the illustrated example, the control device 40 comprises a signal interface 43A for providing control signals to and receiving measurement signals from the system 20. The control device 40 also comprises an input interface 43B for connection to one or more input devices 44 that enable a user to input control data, as well as an output interface 43C for connection to one or more output devices 45 for providing feedback data to the user. For example, the input device(s) 44 may comprise a keyboard, keypad, computer mouse, control button, touch screen, printer, microphone, etc., and the output device(s) 45 may comprise a display device, a touch screen, an indicator lamp, an alarm device, a speaker, etc. The user may be a clinically experienced person, such as a physician or a nurse, or the patient.

It is understood that the control device 40 may be configured to generate further control signals and receive further measurement signals. For example, if the system 20 is part of an HD machine, the control device 40 may generate control signals for further pumps, valves, etc. in the HD machine, as represented by the control signal C4 for the effluent pump 25D in FIG. 1.

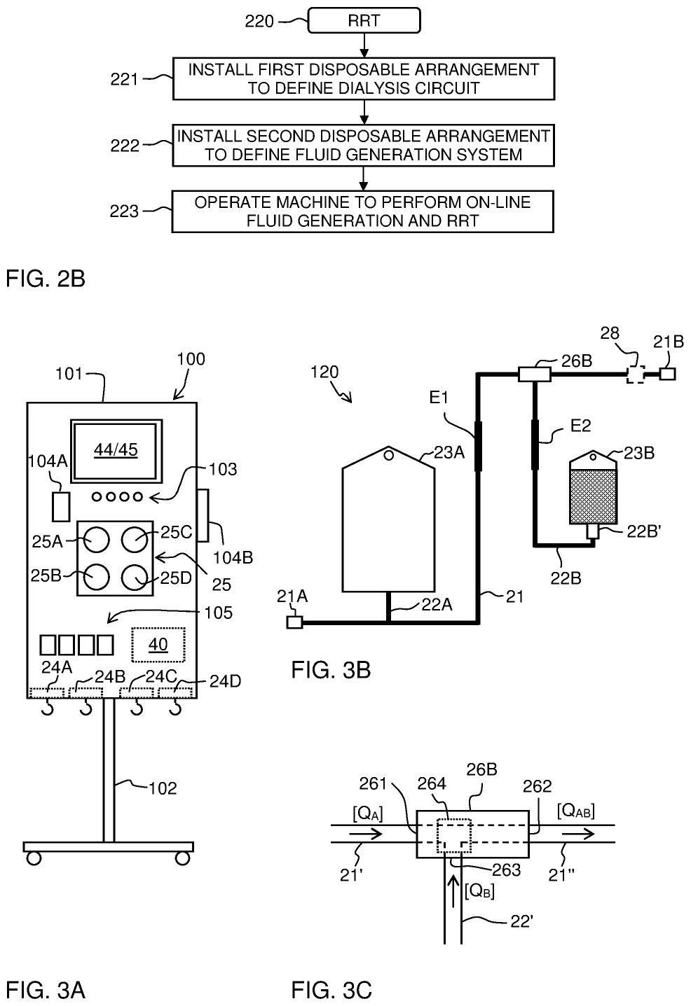

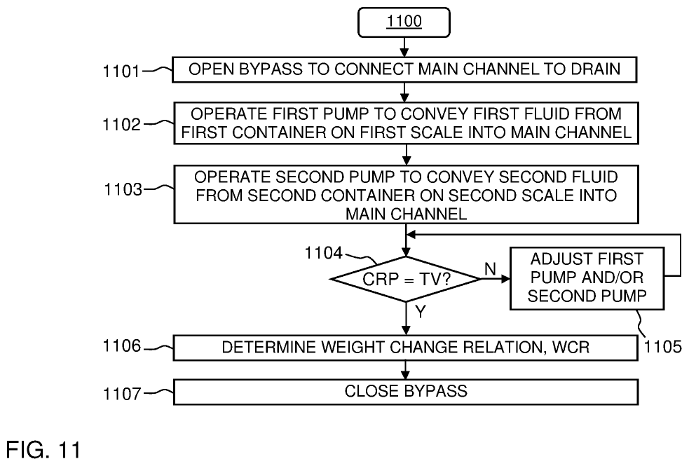

FIG. 2A is a flow chart of an example method 200 of operating the system in FIG. 1 to generate dialysis fluid. The method 200 may be performed by the control device 40 by through control signals C1-C3. The method 200 presumes that water has been admitted into the first container 23A from the source 10, by opening the valve 27. In step 201, pump 25A is operated to pump a first fluid (here, water) from container 23A through the main channel 21. In step 202, pump 25B is operated to pump a second fluid (here, concentrate) from container 23B through the connecting fluid channel 22B into the main channel 21 at junction 26B, to admix the second fluid within the main channel. Step 204 is performed concurrently with steps 201 and 202 to control pumps 25A, 25B, based on the measurement signals S1, S2 from the scales 24A, 24B, to achieve a designated proportion between a first flow rate (Q_{A}) of water into junction 26B and a second flow rate of concentrate (Q_{B}) into junction 26B. The designated proportion results in a desired mixing ratio between concentrate and water. The designated proportion may be determined by the user, calculated by the control device 40 or determined in a dedicated tuning procedure (FIG. 11, below)

The method 200 provides a simple and well-controlled technique of generating dialysis fluid, by step 204 performing open-loop or closed-loop control of the pumps 25A, 25B based on the measurement signals S1, S2.

When the first fluid is water, step 204 effectively dilutes the concentrate from the container 23B to achieve a target composition of the dialysis fluid.

Step 204 may involve determining, from the signals S1, S2 and while the pumps 25A, 25B are operating, a first weight change of the first scale 24A during a first time period, and a second weight change of the second scale 24B during a second time period. Based on the first and second weight changes and the lengths of the first and second time periods, step 204 may determine the weight change per unit time for each of the first and second scales 24A, 24B. The weight changes per unit time represent the mass flow rates Q_{A}, Q_{B}. Step 204 may then set the speeds of the pumps 25A, 25B to achieve a relation between the mass flow rates that corresponds to the designated proportion, for example Q_{A}/Q_{B}=R1, or Q_{A}/Q_{AB}=R1′. Step 204 may perform open-loop control, by calculating a calibration value of the stroke volume for the respective pump 25A, 25B based on S1 and S2, and by setting the speed of the respective pump to fulfil R1 or R1′. Alternatively, step 204 may perform closed-loop control, by controlling the respective pump in view of S1 and S2 to achieve a target value of Q_{A} and Q_{B}, with Q_{A} and Q_{B} fulfilling R1 or R1′. Even higher precision may be achieved by performing step 204 to also account for the measurement signal S3 from sensor 36 (if present), which represents a property of the resulting dialysis fluid. Thus, depending on the measurement signal S3, step 204 may adjust the speed of at least one of pumps 25A, 25B.

Step 204 may also control the pumps 25A, 25B to achieve a given flow rate of the resulting dialysis fluid, Q_{AB}, for example if the system 20 is configured for on-line fluid generation. The control of Q_{AB} may be facilitated by arranging the pump 25A downstream of the junction 26B (cf. FIG. 10).

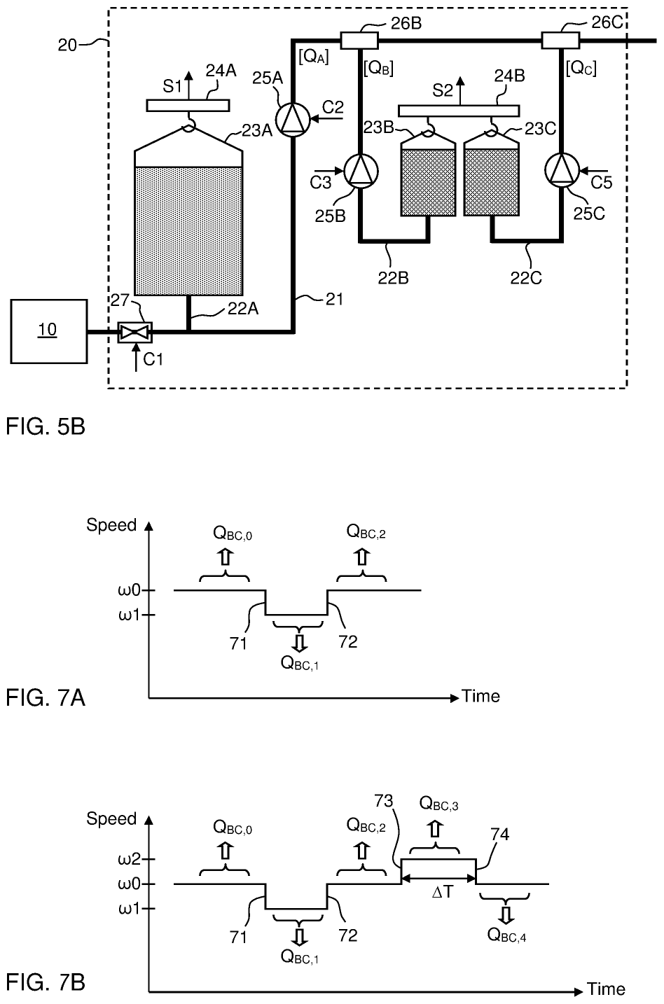

The method 200 is not only applicable to mixing of two fluids but may be extended to mixing of any number of fluids. As an example of this, the method 200 in FIG. 2A includes a step 203, which operates a third pump to pump a third fluid, for example another concentrate, from a third container on the second scale or on a third scale, into the main channel to admix the third fluid in the main channel. Embodiments of step 203, and corresponding modifications of step 204, will be exemplified below with reference to FIGS. 5A-5B.

FIG. 2A also indicates that the method 200 may comprise a step 204A, which requests the user to take a sample of the resulting dialysis fluid at the sampling port (28 in FIG. 1) and to input composition data for the sample. The composition data may be obtained by conventional analysis of the sample, for example to determine its conductivity or the concentration of one or more substances. The request may be generated on the output device 45 (FIG. 1), and the composition data may be input via the input device 44 (FIG. 1). The method 200 may further comprise a step 204B which, upon receiving the composition data, updates the control performed by step 204 to adjust one or more properties of the dialysis fluid. For example, step 204B may compensate for a deviation in the composition of the concentrate in the container 23B from a nominal composition, measurement inaccuracies of the scales 24A, 24B, etc. In some embodiments, the method 200 performs steps 204A-204B whenever the container 23B has been refilled or replaced.

FIG. 2B is a flow chart of an example method 220 for performing RRT by use of an apparatus, for example the above-mentioned monitor. The method 220 is performed by a user of the monitor. In step 221, the user installs a first disposable arrangement on the monitor to define a dialysis circuit. With reference to FIG. 1, the dialysis circuit comprises the dialyzer 30, the effluent line 37, the effluent pump 25D, and an extracorporeal blood circuit that includes the blood lines 34, 35. Such a first disposable arrangement and its use is well-known in the art and will not be described in detail. The first disposable arrangement includes at least the dialyzer 30, the effluent line 37, and a line set that defines the blood lines 34, 35. The line set may also include a drip chamber, one or more access devices on the blood lines 34, 35 for connection to the blood circulatory system of the patient, etc. In step 222, a second disposable arrangement is installed on the monitor to define the fluid generation system 20 and is connected to the source of water (10 in FIG. 1) and to the container of concentrate (23B in FIG. 1) if not already included in the second disposable arrangement. An example of the second disposable arrangement and its installation will be described below with reference to FIGS. 3A-3B. In step 223, the monitor is operated to concurrently perform RRT and generate medical fluid for use in the RRT, for example dialysis fluid. In an alternative, step 221 is omitted, and step 223 operates the monitor to generate medical fluid only. In a further alternative, step 222 is omitted and the system 20 is instead integrated into the monitor.

FIG. 3A shows a schematic example of a monitor 100. The monitor 100 has a chassis 101 mounted on a stand 102, which in this example is provided with wheels to facilitate repositioning of the monitor 100. Scales 24A-24D are arranged in the chassis 101 and connected to projecting hook-like elements, on which containers may be arranged by a user of the monitor 100. A pump arrangement 25 is provided on the chassis 101 to define a plurality of peristaltic pumps 25A-25D, which are accessible to the user. The monitor 100 further comprises a set of sensor ports 103 connected to sensors inside the chassis 101. The sensors may be configured to measure pressure, temperature, conductivity, etc. In the example of FIG. 3A, the monitor 100 further comprises an air detector 104A, a holder 104B for a dialyzer, and a set of clamps 105. A control unit 40 is arranged within the chassis 101 and configured to control the operation of the monitor 100 by obtaining measurement data from the air detector 104A, the sensors and the scales 24A-24D and by selectively actuating the pumps 25A-25D and the clamps 105. A touch screen is connected to the control device 40 and forms a combined input and output device 44/45 for interaction with the user.

It should be emphasized that the monitor 100 in FIG. 3A is merely given as a non-limiting example. The included components may differ, both in functionality and number. However, it is assumed that the monitor 100 comprises at least two scales, at least two peristaltic pumps, and a clamp. These components will implement the scales 24A, 24B, the pumps 25A, 25B and the valve 27 of the system 20 in FIG. 1. This type of monitor is commonly used to treat patients with acute kidney injury, AKI, by so-called acute dialysis. In acute dialysis, the patient is typically continuously treated by RRT and this treatment is commonly known as CRRT. The continuous nature of acute dialysis requires accurate control of fluid removal (“ultrafiltration”). To accurately monitor and control fluid removal, machines for acute dialysis normally have scales for mounting of a container prefilled with dialysis fluid, and an empty container for receiving the effluent.

As noted in the Background section, the distribution and handling of pre-filled containers of dialysis fluid have many inherent drawbacks. These drawbacks may be overcome by use of a disposable arrangement 120 (“disposable”) shown in FIG. 3B. The disposable 120 may be installed on the monitor 100 to define the fluid generation system 20 of FIG. 1. In the example of FIG. 3B, the disposable 120 defines the main channel 21, which extends from an inlet connector 21A to an outlet connector 21B. The inlet connector 21A is configured for connection to the source 10 (FIG. 1), and the outlet connector 21B is configured for connection to the dialyzer 30 or to a sensor port 103 on the monitor 100 (see below). The disposable 120 further comprises or defines the first container 23A, the first connecting fluid channel 22A, the junction 26B, the second connecting fluid channel 22B, and the second container 23B as described with reference to FIG. 1. In an alternative, mentioned above, the inlet connector 21A may instead be arranged on the end of a connecting line which is separate from the main channel 21 and extends from the first container 23A (cf. FIG. 10). The disposable 120 is further provided with a first engagement portion E1 on the main channel 21 and a second engagement portion E2 on the fluid channel 22B. The engagement portions E1, E2 are configured to be engaged by a respective peristaltic pump, as described above. In a variant, the engagement portion E1 is instead located downstream of junction 26B. As shown by dashed lines, the disposable 120 may also include a sampling port 28, as described with reference to FIG. 1. Although not shown, the disposable 120 may also include the sensor 36 and/or the bypass channel 121 (FIGS. 9-10) and/or the effluent line 37 and/or the above-mentioned sterilizing grade filter(s).

In accordance with step 222 of FIG. 2B, the disposable 120 may be installed on the monitor 100 to define the system 20. At this time, the first container 23A is empty, and the second container 23B is filled with concentrate. The disposable 120 may be delivered as a unitary component or in parts that are joined by the user before or during mounting of the disposable 120 on the monitor 100. For example, the second container 23B may be delivered separately and attached by the user on a connector 22B′ on the end of the fluid channel 22B.

The installation of the disposable 120 of FIG. 3B on the monitor of FIG. 3A may involve hanging container 23A on scale 24A, hanging container 23B on scale 24B, attaching connector 22B′ to container 23B, arranging engagement portion E1 on pump 25A, arranging engagement portion E2 on pump 25B, and arranging the main channel 21 in a clamp 105, which thereby operates as valve 27 in FIG. 1. Further, inlet connector 21A is connected to the source 10, which may be separate from the monitor 100, and outlet connector 21B may be connected to the dialyzer 30. If the disposable 120 comprises a bypass channel 121 (FIG. 9), the main channel 21 and the bypass channel 121 may be arranged in two further clamps 105, which thereby form the valve arrangement 27′, 27″ in FIG. 9. If the disposable 120 comprises the sensor 36 (FIG. 1), a wire on the sensor 36 may be connected to a data input port (not shown) on the monitor 100 to transfer signal S3 to the monitor 100. In a variant, if the sensor 36 is available inside the monitor 100, outlet connector 21B may be connected to a dedicated sensor port 103 on the monitor 100, and an outlet port (not shown) on the monitor 100 may be connected in fluid communication with the dialyzer 30. Thereby, the dialysis fluid generated by the system 20 is directed via the sensor port 103 through the sensor 36 and via the outlet port into the first compartment 31 of the dialyzer 30 (FIG. 1).

It is realized that the monitor 100 needs to have enough components to accommodate both the first and the second disposable arrangement. For example, in addition to the components required for installation of the disposable 120 in FIG. 3B, installation of the first disposable arrangement (step 221 in FIG. 2B) may require at least two vacant pumps, which will operate as the effluent pump (cf. 25D in FIG. 1) and a blood pump in the extracorporeal blood circuit, and at least one vacant scale, on which an empty container for receiving the effluent is mounted.

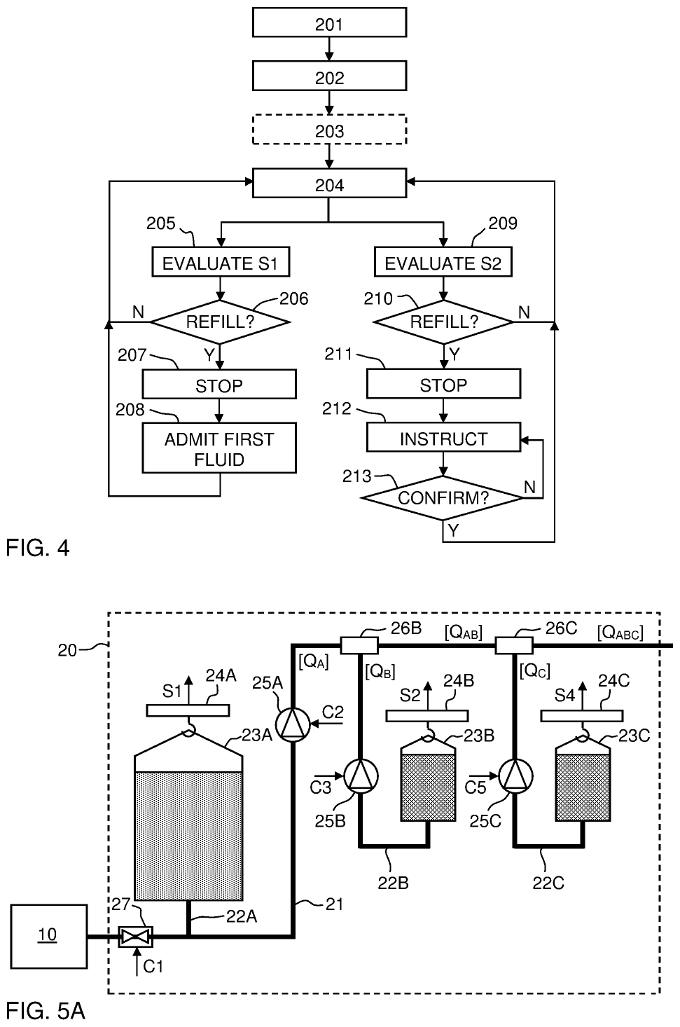

An example operation of the system 20 in FIG. 1, after mounting of the disposable 120 (FIG. 3A) on the monitor 100 (FIG. 3B), will now be described with reference to the flow chart in FIG. 4. The initial state of the system 20 is that the first container 23A is empty and the second container 23B holds a quantity of concentrate. In an initial step, not shown in FIG. 4, valve 27 is opened to admit water into the first container 23A while pump 25A is stopped. Once a predefined amount of water has entered container 23A, as indicated by scale 24A, valve 27 is closed. The system 20 is then operated in accordance with steps 201-204, and optionally steps 204A-204B, as described with reference to FIG. 2A. As noted above, step 204 may operate the pumps 25A, 25B to achieve a target value for the designated proportion between the mass flow rates Q_{A} and Q_{B} and, optionally, a target value for the flow rate of dialysis fluid, Q_{AB}. Either of these target values may be changed at any time during operation. The expected composition of the resulting dialysis fluid may be calculated and displayed to the user on the output device 45 if the control device 40 has information about the composition of the concentrate.

In FIG. 4, the operation of the system 20 involves a first check procedure for replenishing the first container 23A, represented by steps 205-208, and a second check procedure for replacing the second container 23B, represented by steps 209-213.

The first check procedure comprises step 205, which evaluates the measurement signal S1 from scale 24A to detect a need to replenish or refill the container 23A. For example, step 205 may compare the current weight measured by scale 24A to a reference weight, and determine a need for replenishment when the current weight is below the reference weight. The reference weight may be predefined or set in relation to the weight of the container 23A at startup of the system 20, i.e., when empty. If no need for refill is detected, step 206 returns the procedure to step 204. Otherwise, step 206 proceeds to step 207, which stops the pumps 25A, 25B and thereby temporarily suspends the flow of dialysis fluid. If RTT is performed concurrently with the fluid generation, the effluent pump 25D may also be stopped. After step 207, the valve 27 is opened to admit water into the container 23A in step 208. Once a predefined amount of water has entered the container 23A, as indicated by scale 24A, the valve 27 is closed, whereupon the procedure starts the pumps and returns to step 204.

The second check procedure comprises step 209, which evaluates the measurement signal S2 from scale 24B to detect a need to replace the container 23B. For example, step 205 may compare the current weight measured by scale 24B to a reference weight, and determine a need for replenishment when the current weight is below the reference weight. The reference weight may be predefined or set in relation to the weight of the container 23B at startup of the system 20, i.e., when full. If no need for replenishment is detected, step 210 returns the procedure to step 204. Otherwise, step 210 proceeds to step 211, which stops operation in the same way as step 207. After step 211, the user is instructed (step 212), via the output device 45, to disconnect the container 23B and install a new, full container 23B. The system 20 then waits for confirmation by the user via the input device 44. When confirmation is received (step 213), and optionally provided that scale 24B measures a sufficient weight, the procedure starts the pumps and returns to step 204. If the weight measured by scale 24B is too low, or if step 213 waits for too long (time-out), a new instruction may be provided by step 212.

FIG. 3C is a schematic view of the junction 26B as implemented by a 3-way connector, which defines an internal manifold with three ports 261, 262, 263. Sections 21′, 21″ of the main channel 21 are connected to ports 261, 262, and a section 22′ of the connecting fluid channel 22B is connected to port 263. The sections 21′, 21″, 22′ may be permanently or releasably connected to the ports 261, 262, 263 and may be configured as tubing. Incoming flows (Q_{A}, Q_{B}) at ports 261, 263 meet in the internal manifold and form a combined flow (Q_{AB}) through port 262. The 3-way connector 26B comprises a device 264 for promoting or enhancing mixing of the incoming flows. This mixing-enhancement device 264 may be configured to increase the Reynolds number of the combined flow and/or either of the incoming flows. In some embodiments, the mixing-enhancement device 264 may be configured to generate or increase turbulence downstream of the device 264. In some embodiments, the device 264 defines a constriction, which may be located anywhere within the internal manifold to form a passage of reduced and then expanded the cross-section. A non-limiting example of a 3-way connector with a mixing-enhancement device is disclosed in WO2009/030973, which is incorporated herein by reference. Although this known 3-way connector is configured for infusion of a solution into blood, the skilled person can adapt the teachings to fluids of lower viscosity and to the flow rates used in the system 20. In a variant, the mixing-enhancement device 264 may be separate from and located downstream of the junction 26B. Such a separate device 264 may be of the same configuration as the integrated device described above. Alternatively, the separate device 264 may be configured as a conventional static mixer, or a recirculation circuit in which the combined flow is circulated to promote mixing before being conveyed to the dialyzer.

In some embodiments, the mixing-enhancement device 264 is configured to ensure efficient and immediate mixing of the fluids. In other embodiments, a lesser or slower degree of mixing may be acceptable, as long as the fluids are sufficiently mixed when reaching the dialyzer 30, for example depending on the distance between the junction 26B and the dialyzer 30, or on the type of RRT. For example, it is currently believed that CRRT is more tolerant to incomplete mixing. However, the required degree of mixing may also depend on the chemical properties of the fluids. For example, if two concentrates are mixed with water, as will be exemplified with reference to FIGS. 5A-5B, local chemical instability may occur in case of incomplete mixing. For example, bicarbonate concentrate may be prone to precipitate with calcium from the other concentrate if mixing is incomplete.

FIG. 5A depicts a system 20 configured to generate a dialysis fluid by mixing three fluids. The following description will focus only on differences over the system 20 in FIG. 1. Compared to FIG. 1, the system 20 comprises a third scale 24C, a third container 23C on the third scale 24C, a third connecting fluid channel 22C that extends between the third container 23C and a second junction 26C on the main channel 21 downstream of the junction 26B. A fluid pump 25C is arranged to pump a third fluid from the third container 23C towards the junction 26C, and thus into the main channel 21. The junction 26C receives a combined flow of the first and second fluids, into which the third fluid is admixed. In the following, the flow rate of the combined flow from junction 26B is denoted Q_{AB}, the flow rate of the third fluid is denoted Q_{C}, and the flow rate of the resulting dialysis fluid is denoted Q_{ABC}, as indicated within brackets in FIG. 5A. The junction 26C may have the same configuration as the junction 26B, for example as described with reference to FIG. 3C. In the following, it is assumed that the first fluid is water, and that the second and third fluids are first and second liquid concentrates that are components of a dialysis fluid. Any concentrates known in the art may be used.

As mentioned above, the method 200 in FIG. 2 is also applicable to mixing of three fluids. In the example of FIG. 5A, step 203 operates pump 25C to pump the second concentrate from container 23C through the third connecting fluid channel 22C into the main channel 21 to admix the second concentrate within the main channel 21. In addition to controlling pumps 25A, 25B to achieve a first proportion between Q_{A} and Q_{B}, step 204 controls pump 25C, based on signal S4 from scale 24C, to achieve a second proportion between the flow rate (Q_{B}) of the first concentrate into the first junction 26B and the flow rate (Q_{C}) of the second concentrate into the second junction 26C. The controlling of pump 25C may be performed by analogy with the controlling of pumps 25A, 25B. It should be noted that pump 25C may be set in relation to either of pumps 25A, 25B to achieve the second proportion. Ultimately, step 204 controls pumps 25A, 25B, 25C to achieve a relation (mixing ratio) between Q_{A}, Q_{B}, Q_{C} that matches a recipe for the dialysis fluid. As understood, step 204 may also control pumps 25A, 25B, 25C to achieve a given flow rate of the resulting dialysis fluid, Q_{ABC}, for example if the system 20 is configured for on-line fluid generation.

To operate the system 20 in FIG. 5A, the control device 40 of FIG. 1 is further configured to receive the measurement signal S4 from the scale 24C and to output the control signal C5 for the pump 25C. Further, the operation in accordance with FIG. 4 may include a third check procedure, which corresponds to the second check procedure but evaluates signal S4 from scale 24C to detect a need to replace container 23C.

It is also to be understood that the system 20 in FIG. 5A may be implemented by a modified version of the disposable arrangement 120 in FIG. 3B (“expanded disposable”). Compared to the disposable in FIG. 3B, the expanded disposable further comprises the fluid channel 22C, the second junction 26C, and a third engagement portion on the fluid channel 22C. The third engagement portion is configured to be engaged with pump 25C. The third container 23C, filled with the second concentrate, may be connected or connectable to the fluid channel 22C.

In a variant of the expanded disposable, and the system in FIG. 5A, the fluid channel 22C is fluidly connected to the fluid channel 22B or the first junction 26B, and the second junction 26C is omitted.

The system in FIG. 5A is a simple and straight-forward extension of the system in FIG. 1. However, it presumes the availability of an additional scale 24C. There may be situations in which the additional scale is not available, for example if the expanded disposable is to be arranged on a machine that only has two vacant scales for fluid generation.

FIG. 5B shows an example system 20 that only requires two scales to mix three fluids. The system in FIG. 5B differs structurally from the system in FIG. 5A only in that the second and third containers 23B, 23C are arranged on the second scale 24B. Thus, the measurement signal S2 of scale 24B represents the combined weight of the containers 23B, 23C.

The method 200 is also applicable to the system 20 of FIG. 5B. Step 203 operates the third pump 25C as described above for the system in FIG. 5A. In addition to controlling pumps 25A, 25B to achieve the first proportion between Q_{A} and Q_{B}, step 204 controls pump 25C to achieve a second proportion between the flow rate (Q_{B}) of the first concentrate into the first junction 26B and the flow rate (Q_{C}) of the second concentrate into the second junction 26C. In one example, step 204 may control pump 25A to generate a flow rate Q_{A} that will result in a desired (target) flow rate of dialysis fluid, Q_{ABC}, when the second and third pumps 25B, 25C are controlled to achieve the first and second proportions. Step 204 may further jointly control the second and third pumps 25B, 25C to generate Q_{B} and Q_{C} in accordance with the first and second proportions, based on the stroke volume of the respective pump 25B, 25C. Specifically, step 204 may maintain a relative speed between the pumps 25B, 25C that results in the second proportion between their expected flow rates (given by the product of the speed and stroke volume for each pump). Step 204 may further set the speeds of the pumps 25A, 25B, while maintaining the relative speed between the pumps 25B, 25C, to achieve the first proportion between Q_{A} and Q_{B}. The stroke volumes may be predefined or measured for the respective pump 25B, 25C. For example, a calibration value of the stroke volume may be determined, during a calibration procedure, by operating only one of the pumps 25B, 25C to perform a number of strokes per unit time (“pumping rate”) and determining the corresponding weight change from the signal S2 (“mass flow rate”). As used herein, “stroke volume” may be given in terms of volume or mass per pumping stroke. In the foregoing example, the stroke volume by mass may be calculated by dividing the mass flow rate by the pumping rate.

Step 204 may perform closed-loop control, by controlling pump 25A to achieve a target value of Q_{A} in view of S1, and by jointly controlling pumps 25B, 25C in view of S2 to achieve a target value of Q_{B}+Q_{C}. It is also conceivable for step 204 to account for the measurement signal S3 from sensor 36 (if present). Thus, depending on the measurement signal S3, step 204 may adjust the speed of at least one of pumps 25A, 25B, 25C.

The systems in FIGS. 5A-5B may be modified to instead locate the pump 25A downstream of the junction 26C. Thereby, the pump 25A defines Q_{ABC} and also indirectly defines Q_{A}. It is realized that the method 200 is equally applicable with this placement of the pump 25A to control the pumps 25A, 25B, 25C to achieve any desired mixing ratio between Q_{A}, Q_{B}, Q_{C}.

To operate the system 20 in FIG. 5B, the control device 40 of FIG. 1 is further configured to output the control signal C5 for pump 25C. The first and second check procedures in FIG. 4 may be used also in the system of FIG. 5B. For example, if the second and third containers 23B, 23C are expected to be depleted at approximately the same time, the second check procedure may infer and signal a need to replace both containers 23B, 23C when the current weight measured by scale 24B falls below a reference weight. Alternatively, steps 209-210 of the second check procedure may be modified to calculate the accumulated amount of fluid that has been pumped from the respective container 23B, 23C and detect a need to refill one of the containers when the accumulated amount for this container exceeds a reference amount. The accumulated amounts may be calculated by dead reckoning, for example by counting the number of strokes and multiplying the number of strokes with the stroke volume.

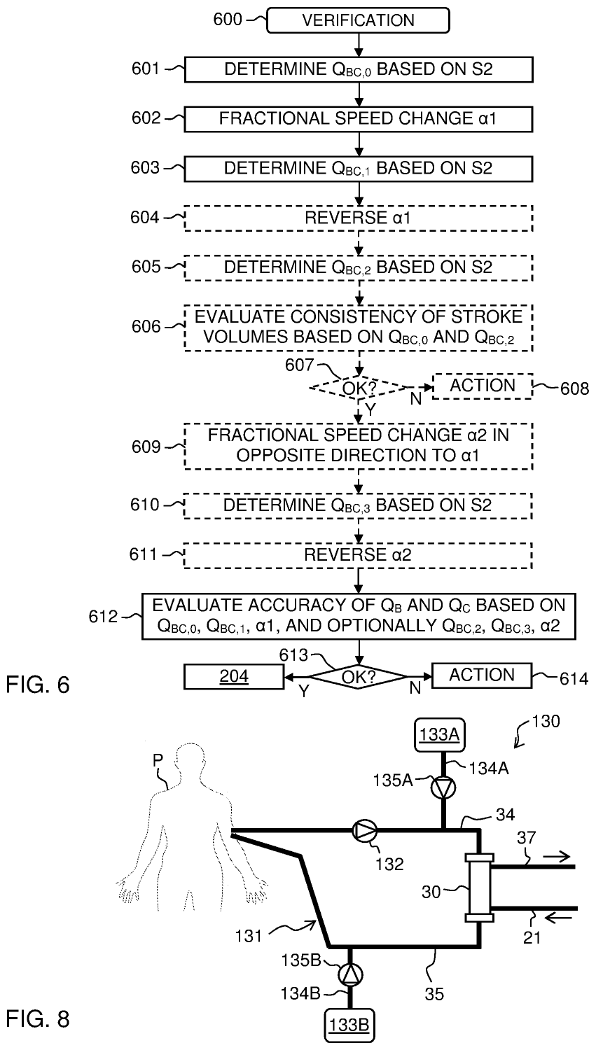

In FIG. 5B, when the flow rates Q_{B} and Q_{C} are controlled by setting the speeds of the pumps 25B, 25C based on their stroke volumes, it may be desirable to verify that the flow rates Q_{B} and Q_{C} match their respective target value. FIG. 6 is a flow chart of a verification procedure 600 that may be included in the method 200 of FIG. 2A and performed at least once or intermittently during fluid generation to quantify the flow rates Q_{B} and Q_{C}. Optional steps are indicated by dashed lines in FIG. 6. In the following, the combined flow rate of the second and third fluids (Q_{B}+Q_{C}) is designated by Q_{BC}. FIGS. 7A-7B serve to exemplify the speed of one of the pumps 25B, 25C over time during the procedure 600.

At start of the procedure 600, the pumps 25B, 25C are operated at a respective initial speed, represented as 00 for one pump in FIG. 7A, resulting in flow rates Q_{B,0} and Q_{C,0}. In step 601, the combined flow rate Q_{BC} is determined from the measurement signal S2, based on the measured weight change over time. This results in an initial combined flow rate, Q_{BC,0}. In step 602, the speed of one of the pumps 25B, 25C (“selected pump”) is changed by a predefined fractional amount, α1. This is seen as a step change 71 from ω0 to ω1 in FIG. 7A. The speed may be decreased, as shown in FIG. 7A, or increased. The speed of the other pump is fixed throughout the procedure 600. The fractional amount may be any value, for example in the range of 1-20%. In step 603, the combined flow rate Q_{BC} is again determined from the measurement signal S2, resulting in a first subsequent combined flow rate, Q_{BC,1}. It may be preferable to then reverse the fractional change, by step 604, which thereby returns the selected pump to its initial speed ω0, as shown by step change 72 in FIG. 7A. This limits the impact of the procedure 600 on the composition of the dialysis fluid. The procedure 600 may then proceed to step 612, which evaluates the pumping accuracy, i.e. the accuracy of Q_{B} and Q_{C} based on Q_{BC,0}, Q_{BC,1} and α1. The evaluation is based on the following set of equations, assuming that pump 25B is the selected pump:

$${Q}_{BC , 0} = ω 0 \cdot {V}_{B} + {ω}_{C} \cdot {V}_{C}$$

$${Q}_{B C , 1} = ω 1 \cdot {V}_{B} + {ω}_{C} \cdot {V}_{C} = α 1 \cdot ω0 \cdot {V}_{B} + {ω}_{C} \cdot {V}_{C}$$

with V_{B} being the stroke volume of pump 25B, V_{C} being the stroke volume of pump 25C, and ω_{C} being the speed of pump 25C. These equations assume that the stroke volumes V_{B}, V_{C} are not changed between steps 601, 603 and may be rearranged as: Q_{BC,0}−Q_{BC}1=ω0. V_{B}. (1-α1)=Q_{B,0}·(1-α1). Thus, the flow rates Q_{B} and Q_{C} at the start of the procedure 600 may be calculated by step 612 as:

$$\frac{{Q}_{B , 0} = ( Q {BC , 0}_{} - {Q}_{BC , 1} )}{1 - α 1}$$

$${Q}_{C , 0} = ( Q {BC , 0}_{} - {Q}_{B , 0} )$$

A corresponding set of equations may be given if pump 25C is instead the selected pump.

Step 613 then evaluates the resulting values of Q_{B,0} and Q_{C,0} with respect to target values. If a deviation of sufficient magnitude is not found, step 613 proceeds to step 204 (FIG. 2A). Otherwise, if the deviation exceeds a limit value, step 613 proceeds to step 614, which may modify the speed of the pumps 25B, 25C to better match Q_{B} and Q_{C} to the target values, and then possibly jointly modify the speeds of pumps 25A, 25B, 25C to achieve the target value of Q_{ABC}. For example, step 614 may calculate updated values of the stroke volumes V_{B}, V_{C} and set the speeds of the pumps 25B, 25C to generate the target values of Q_{B}, Q_{C} for the updated stroke volumes. If the deviation is excessive, step 614 may stop the fluid generation and/or output a warning for the user.

The procedure 600 may comprise steps 605-608, which serve to detect if step 602 changes the stroke volumes V_{B}, V_{C}. In step 605, after the reversal by step 604, the combined flow rate Q_{BC} is determined from the measurement signal S2, by analogy with step 601. This results in a second subsequent combined flow rate, Q_{BC,2}, as shown in FIG. 7A. Step 606 evaluates the consistency of the stroke volumes by comparing Q_{BC,2} and Q_{BC,0}. Step 606 is based on the understanding that a change in stroke volume is likely to emerge as a hysteresis in Q_{BC}. If a deviation is found that exceeds a limit value, step 607 proceeds to step 608 which may stop the fluid generation and/or output a warning for the user.

The procedure 600 may comprise step 609 and 611, which serve to compensate for the change in composition of the dialysis fluid caused by steps 602 and 604. In step 609, the speed of the selected pump is changed by a second fractional amount, α2. This is seen as a step change 73 from ω0 to ω2 in FIG. 7B. The step change in step 609 is made in the opposite direction to the step change in step 602. Thus, if step 602 increases the speed, step 609 decreases the speed, and vice versa. Step 611 reverses the second fractional change to return the selected pump to its original speed ω0, as shown by step change 74 in FIG. 7B. Steps 609-611 are included to provide the selected pump with a mean pumping speed that is equal to ω0 over the verification procedure 600 as a whole.

The procedure 600 may also comprise step 610, which determines the combined flow rate Q_{BC} from the measurement signal S2, by analogy with step 601. This results in a third subsequent combined flow rate, Q_{BC,3}, as shown in FIG. 7B. Step 611 may be modified to also account for Q_{BC,2}, Q_{BC,3} and α2, thereby providing an overdetermined system of equations that may improve the accuracy of steps 612, 614.

It may be noted that the compensation as depicted in FIG. 7B and described with reference to steps 609 and 611 in FIG. 6 is merely an example. In one alternative, the pumping speed of the selected pump varies during the compensation. In another alternative, steps 604-608 are omitted and the compensation is made from ω1 in FIG. 7B. Generally, the compensation may be seen to involve changing the pumping speed of the selected pump for a period of time (ΔT in FIG. 7B) so as to counteract an increase or decrease in the amount of fluid pumped by the selected pump as a result of the first fractional change by step 602.

In some embodiments, the system 20 may be operated to direct the fluid flow into a bypass channel 121 (FIG. 9) whenever the composition of the dialysis fluid is deemed likely to deviate from the target composition, for example during transitory phases such as startup or when the target composition is changed drastically. For example, the fluid flow may be directed through the bypass channel 121 during the procedure 600. Further, the fluid flow may be directed into the bypass channel 121 during the above-mentioned calibration procedure for determining the calibration value of the stroke volume of the respective pump 25B, 25C.

FIG. 8 is included to provide a non-limiting example of an extracorporeal blood circuit (EBC) 130 that may be used in combination with the fluid generation system 20. The EBC 130 may, for example, be used in CRRT. In FIG. 8, the EBC 130 is connected to a patient P at a blood withdrawal end and a blood return end. The connections may be performed by any conventional device, such as a needle or catheter. The EBC 130 comprises a disposable 131 which is mounted to pumps 132, 135A, 135B on an RRT apparatus (cf. 100 in FIG. 3A). The disposable 131 comprises blood lines or tubing that define a blood withdrawal path 34 and a blood return path 35. A dialyzer 30 is connected between the withdrawal and return paths 34, 35. A blood pump 132 is arranged to draw blood from the patient P and pump the blood via the blood compartment of the dialyzer 30 and back to the patient P. The dialyzer 30 is connected to receive dialysis fluid on fluid path 21 and to output effluent on fluid path 37. In the illustrated example, the EBC 130 further comprises a first source 133A of replacement fluid which is connected by a fluid line 134A to the withdrawal path 34 intermediate the blood pump 132 and the dialyzer 30. A fluid pump 135A is arranged to pump the replacement fluid from the source 133A into the withdrawal path 34. The EBC 130 further comprises a second source 133B of replacement fluid which is connected by a fluid line 134B to the return path 35. A fluid pump 135B is arranged to pump the replacement fluid from the source 133B into the return path 35. In the example of CRRT, the EBC 130 may also comprise an arrangement for infusion of an anti-coagulant agent, for example citrate or heparin, or an arrangement for infusion of a calcium-containing solution.

It is understood that the fluid generation system 20 as described herein may be connected to provide the dialysis fluid to the dialyzer 30 in FIG. 8. Alternatively or additionally, the replacement fluid may be generated by such a system 20.

As noted, FIG. 8 is merely an example, and the EBC 130 may include other conventional components, such as clamps, pressure sensors, air detector, drip chamber, etc. Also, the pre-infusion and/or post-infusion of replacement fluid may be omitted.

There are commercially available concentrates that may be used in the fluid generation system 20 as described herein.

In some embodiments, dialysis fluid for treatment of patients with chronic kidney disease (CKG) by hemodialysis, hemofiltration or hemodiafiltration is generated by mixing a single concentrate with water at a dilution ration of 10-50 by volume. In a non-limiting example, the single concentrate comprises lactate, sodium, potassium, calcium, magnesium, glucose and chloride. Such a concentrate is, for example, commercially available for the PureFlow SL system from NxStage. Alternatively, the dialysis fluid may be generated by mixing two concentrates with water. For example, a bicarbonate concentrate and an acid concentrate may be mixed with water at a dilution ratio of 10-50. Such concentrates are commercially available and well-known in the art. In a non-limiting example, the bicarbonate concentrate comprises bicarbonate, and the acid concentrate comprises sodium, potassium, calcium, magnesium, glucose, acetate and chloride. In some acid concentrates, acetate is replaced or supplemented by another acid, for example citric acid.

In some embodiments, dialysis fluid for CRRT treatment of patients with acute kidney injury (AKI) is generated by mixing at least one concentrate with water. In a non-limiting example, such a dialysis fluid comprises bicarbonate, sodium, potassium, calcium, magnesium, phosphate, glucose, acetate and chloride. In one example, a base concentrate and an electrolyte concentrate may be mixed with water to form the dialysis fluid. For example, the base concentrate may be an alkaline hydrogen carbonate solution, and the electrolyte concentrate may be an acidic glucose-based electrolyte solution.

In some embodiments, dialysis fluid for use in peritoneal dialysis (PD) is generated by mixing at least one concentrate with water. Example compositions of PD concentrates, to be mixed with water individually or in combination, are disclosed in US2018/0021501 and WO2017/193069, which are incorporated herein by reference.

The foregoing disclosure is equally applicable to mixing of more than three fluids for generation of a medical fluid. For example, the system 20 in FIG. 5A may be further extended to include a further scale for each additional container to be installed. It is also conceivable to arrange two containers on one scale in FIG. 5A, by analogy with FIG. 5B. In a further variant, more than two containers may be arranged on one scale in FIG. 5A or FIG. 5B.

Reverting to FIG. 1, the control device 40 operates the system 20 based on input control data that is received via the input interface 43B. The input control data may be at least partly manually entered by the user. In some embodiments, the input control data is indicative of the concentrate(s) installed in the system, and a target composition of the medical fluid to be generated. For example, the input control data may identify a nominal or actual composition of the respective concentrate, thereby allowing the control device 40 to determine the above-mentioned proportion(s) between concentrates and water to achieve the target composition. In some embodiments, the input interface 43B is connected to a dedicated reader device (cf. 44), and the nominal or actual composition is given as the reader identifies a bar code or RFID tag on the respective container or performs optical character recognition (OCR) of a label on the respective container. In an alternative embodiment, the respective proportion between the fluids is directly entered by the user. The input control data may also indicate a target value of the flow rate of the medical fluid to be generated. If the system 20 is operated for on-line fluid generation, this target value may be given by a setting of the RRT or a signal indicative of the current consumption of the medical fluid by the RRT.

It is also to be understood that safety features may be included in the fluid generation system 20. Such safety features comprise installing a second independent system of scales to enable detection of malfunctions, using keyed connectors to prevent misconnection of containers, using different weights of different containers to enable detection of misconnection, using color coding to facilitate correct connection of containers, etc.

As noted above, the system 20 may include a sensor 36 for measuring the conductivity of the generated medical fluid (cf. FIG. 1). The fluid pumps in the system 20 may be controlled to achieve a designated proportion or ratio between the flow rates based on the measurement signals from the scales, and the measured conductivity may be used by a protective function which is configured to detect deviations and take protective measures. Alternatively, the fluid pumps in the system 20 may be controlled to achieve the proportion(s) based on the measured conductivity, and the measurement signals from the scales may be used by the protective function. The provision of the sensor 36 allows the system 20 to detect if an erroneous container has been installed, as well as prevent delivery of a medical fluid with major error in composition.

As mentioned with reference to FIG. 2A, the method 200 may comprise a step 204A that involves the use of a sampling port 28 (cf. FIG. 1). In on-line generation, step 204A may instruct the user, for example whenever a new container has been installed in the system 20, to take a sample of the medical fluid and analyze the sample for its content of one or more electrolytes (e.g., sodium, potassium, bicarbonate, etc.), or one or more additives such as glucose. This composition check request might be ignored or omitted if successive containers pertain to the same batch of concentrate. Step 204A may require the user to input the analysis results within a time frame that may be fixed or adjustable, for example within 30-120 minutes after installation of a new container. An adjustable time frame may be set in dependence of dialysis dose, with a larger dialysis dose resulting in a shorter time frame. If the content is found to deviate significantly from the expected composition, the method 200 may interrupt/suspend RRT and request the user to check the installed concentrate(s) for correctness. If the deviations are smaller, the method 200 may instruct the user to take and analyze a new sample. If the deviations persist in the new sample, the method 200 may adjust the proportion(s) in step 204B. If the deviations are not found in the new sample, the method 200 may proceed to use its current settings for generating the medical fluid.

The systems and methods for fluid generation described herein are not limited to HD but are applied to any type of RRT. FIG. 10 shows an example system 20 for generation of medical fluid for any type of RRT. The illustrated system 20 is configured to generate the medical fluid by mixing two fluids, but may be extended to admix further fluid(s) if needed, by analogy with FIG. 5A or FIG. 5B. Components in FIG. 1 and FIG. 10 are identical insofar they are assigned the same reference numerals. The description will not be repeated for such components.

In FIG. 10, the outlet of the main channel 21 is fluidly coupled to a receiving device 30′. The receiving device 30′ is arranged to receive the medical fluid that is generated in the main channel 21 when the system 20 is operated according to the method 200 in FIG. 2A. In some embodiments, the medical fluid is a dialysis fluid for use in extracorporeal blood therapy, such as HD or HDF, and the receiving device 30′ comprises the dialyzer 30 (FIG. 1) and further conventional components. In some embodiments, the medical fluid is a replacement fluid for use in HF or HDF, and the receiving device 30′ comprises an infusion port (not shown) in the withdrawal path 34 and/or the return path 35 (FIG. 1). In some embodiments, the medical fluid is a dialysis fluid for use in PD, and the receiving device 30′ comprises a disposable or reusable fluid circuit, which is attached to a PD cycler. It is also conceivable that the receiving device 30′ corresponds to the peritoneal cavity as such. In some embodiments, which are applicable to all types of RRT, the receiving device 30′ is a reservoir for collecting the medical fluid for subsequent use in RRT. In such embodiments, the medical fluid is typically not generated on-demand. The reservoir may or may not be connected to or part of an apparatus for RRT. As indicated by a dashed line, an effluent channel 37 may extend from the receiving device 30′ to drain 38, for example to dispose of effluent generated in HD, HDF, HF or PD.

Like in FIG. 9, the system 20 of FIG. 10 comprises a bypass channel 121, which defines a fluid path from the main channel 21 to the drain 38. The bypass channel 121 is connected to the main channel 21 upstream of the receiving device 30′. A valve arrangement 27A, which corresponds to the valves 27′, 27″ in FIG. 9, is operable to selectively direct a fluid flow in the main channel 21 into the bypass channel 121 instead of into the receiving device 30′. The valve arrangement 27A is operated by a control signal C6 from the control device 40 (FIG. 1). A CRP sensor 36 is arranged in the bypass channel 121 to measure a composition-related parameter (CRP) of the passing fluid and generate a corresponding measurement signal S3. The CRP may represent conductivity, or equivalently resistivity. In a variant, the CRP represents the concentration of a substance in the fluid, specifically a substance that is present in fresh medical fluid, for example bicarbonate or an electrolyte such as sodium, potassium, calcium, magnesium, chloride, etc. If the medical fluid is generated for use in PD, the substance may alternatively be an osmotic agent such as glucose. In a further alternative, the CRP may represent the concentration of hydrogen ions, for example in the form of a pH value. Any sensor designated by reference numeral 36 herein may be a CRP sensor.

Reverting to the method 200 in FIG. 2A, the composition of the medical fluid that is generated by the mixing of fluids in the main channel 21 is dependent on the accuracy of the designated proportion(s) between the flow rates of the fluids. As noted above, the proportion(s) may be calculated by the control device 40 based on nominal or actual compositions of the included fluids, to achieve a target composition of the medical fluid. Alternatively, the predefined proportion(s) may be directly entered by the user into the control device 40. However, it may be desirable to automatically determine the proportion(s) for the actual fluids that are installed in the system. Such automatic determination is enabled by the system 20 in FIG. 10, by use of the bypass channel 121 and the CRP sensor 36.

FIG. 11 is a flowchart of an example tuning procedure 1100 for automatic determination of the proportion between the flow rates Q_{A}, Q_{B} in FIG. 10. The tuning procedure 1100 is performed by the control device 40. In step 1101, the valve arrangement 27A is operated to close the main channel 21 and open the bypass channel 121. Fluid is thereby directed from the main channel 21 via the bypass channel 121 to the drain 38, while passing the CRP sensor 36. In step 1102, pump 25A is operated to convey the first fluid (water) from the first container 23A via the main channel 21 into the bypass channel 121. In FIG. 10, it is presumed that pump 25B is occluding and thereby inherently blocks fluid channel 22B when not activated. If necessary, a controllable on/off valve (not shown) may be arranged along fluid channel 22B to selectively block the flow of second fluid from the second container 23B. In step 1103, pump 25B is operated to convey the second fluid (concentrate) from the second container 23B via the main channel 21 into the bypass channel 121. At this time, a mixture of the first and second fluids passes through the CRP sensor 36. In step 1104, the signal S3 from the CRP sensor 36 is evaluated for determination of a current CRP value, and the current CRP value is compared to a target CRP value, TV, which defines a required property of the medical fluid to be generated. TV may be predefined and stored in internal memory 42 of the control device 40 or be entered by a user via the input device 44 (cf. FIG. 1). If the current CRP value is found to deviate from TV, the speed of pump 25A and/or pump 25B is adjusted in step 1105. Steps 1104-1105 are repeated until the current CRP value matches TV. Steps 1104-1105 thus define a tuning operation. In some embodiments, the pump 25A may be adjusted in step 1105 to achieve a desired flow rate Q_{AB} in the main channel 21. When the current CRP value matches TV, a weight change relation (WCR) is determined in step 1106 by use of the signals S1, S2 from the scales 24A, 24B. WCR corresponds to the designated proportion and is determined as the relationship between the weight change per unit time for container 23A and the weight change per unit time for container 23B when the current CRP value matches TV. The weight change per unit time may be determined by operating any conventional differentiation algorithm on the respective signal S1, S2. As the WCR is determined, the medical fluid is generated in the main channel 121. Thus, in step 1107, the valve arrangement 27A may be operated to close the bypass channel 121 and open the main channel 21 to direct medical fluid into the receiving device 30′. It may be noted that the tuning procedure 1100 may be performed as part of the method 200, with steps 1101-1102 being performed in step 201, step 1103 being performed in step 202, and steps 1104-1107 being performed as part of step 204. Subsequent to step 1107, the control device 40 may continuously control the speeds of the pumps 25A, 25B so that the signals S1, S2 fulfil WCR and medical fluid of the desired composition is generated.

In a variant, pumps 25A, 25B are deactivated after step 1106, whereupon step 1107 may or may not be performed. The method 200 is then performed at a later time, using WCR as the designated proportion. At startup of the method 200, the valve arrangement 27A may be operated to direct the fluid to drain 38 via the bypass channel 121 until step 204 achieves the designated proportion (WCR). Then, the valve arrangement 27A may be operated to close the bypass channel 121 and direct medical fluid into the receiving device 30′. Optionally, the medical fluid is only directed into the receiving device 30′ if the current CRP value, given by signal S3, matches TV.

The tuning procedure 1100 in FIG. 11 may be expanded if the medical fluid is generated by mixing more than two fluids, for example three fluids as shown in FIGS. 5A-5B. For example, steps 1102-1106 may be repeated for another combination of the available fluids, resulting in determination of a second WCR, which forms the above-mentioned second proportion.

In the technique proposed herein, two or more constituent fluids of the medical fluid are supplied in adequate amounts for mixing in the main channel based on measurement signals from scales associated with containers that hold the respective constituent fluid. This obviates the need for continuous feedback from a CRP sensor to ensure that the medical fluid is generated with a correct composition. In the example of FIG. 10, a CRP sensor 36 is instead arranged in the bypass channel 121 and is only exposed to the constituent fluids during the tuning procedure 1100. Thereby, the medical fluid does not pass the CRP sensor 36 on its way to the receiving device 30′ and is thus not exposed to any microorganisms that may be present in the CRP sensor 36. This mitigates the need for intermittent disinfection of the CRP sensor 36, as well as the need to intermittently replace the CRP sensor 36. Further, by limiting its exposure to the medical fluid, fouling of the CRP sensor 36 is reduced, for example in terms of scaling. Thereby, the operative life of the CRP sensor 36 is extended, and it may even be possible to use a permanent CRP sensor. Since CRP sensors generally are expensive, significant cost savings are possible.

For this reason, the CRP sensor 36 may be releasably connected to a disposable arrangement in the system 20, so that the CRP sensor 36 is re-used while disposable arrangements are discarded between treatments. In the system of FIG. 10, the disposable arrangement includes the first container 23A and the fluid channels 29, 21, 22B and 121. Further, the disposable arrangement comprises an inlet connector 21A for connection to the source 10, an outlet connector 21B for connection to the receiving device 30′, a terminal connector 21C on the bypass channel 121 for connection to the CRP sensor 36, and an inlet connector 22B′ for connection to the second container 23B. Although not shown in FIG. 10, the disposable arrangement may comprise engagement portions for engagement with the pumps 25A, 25B (cf. E1, E2 in FIG. 3B).

In a variant of the system 20 in FIG. 10, the CRP sensor 36 is instead located in the main channel 21 upstream of the valve arrangement 27A. The tuning procedure 1100 in FIG. 11 is equally applicable to this variant. However, as understood from the above, the operative life of a CRP sensor 36 may be more limited at this location.

While the subject of the present disclosure has been described in connection with what is presently considered to be the most practical and preferred embodiments, it is to be understood that the subject of the present disclosure is not to be limited to the disclosed embodiments, but on the contrary, is intended to cover various modifications and equivalent arrangements included within the spirit and the scope of the appended claims.

In the following, a set of clauses are recited to summarize some aspects and embodiments of the invention as disclosed in the foregoing.

C1. A method of generating a medical fluid for use in treatment of blood by renal replacement therapy, said method comprising: operating (201) a first pump (25A) to pump a first fluid from a first container (23A) arranged on a first scale (24A), through a first fluid channel (21), the first fluid being a component of the medical fluid; operating (202) a second pump (25B) to pump a second fluid from a second container (23B) arranged on a second scale (24B), through a second fluid channel (22B) into the first fluid channel (21) at a junction (26B) in the first fluid channel (21), to admix the second fluid within the first fluid channel (21), the second fluid being a component of the medical fluid; and controlling (204) the first and second pumps (25A, 25B), based on first and second output signals (S1, S2) from the first and second scales (24A, 24B), to achieve a first proportion between a first flow rate of the first fluid into the junction (26B) and a second flow rate of the second fluid into the junction (26B).

C2. The method of C1, wherein the first and second fluid channels (21, 22B) and the first and second containers (23A, 23B) are combined to form a disposable arrangement (120), which is replaced during the renal replacement therapy or discarded when the renal replacement therapy is completed.

C3. The method of C1 or C2, wherein said controlling (204) comprises: determining, based on the first and second output signals (S1, S2), a first weight change per unit time for the first scale (24A) and a second weight change per unit time for the second scale (24B), and operating the first and second pumps (25A, 25B) to achieve the first proportion between the first and second weight changes per unit time.

C4. The method of any preceding clause, further comprising: detecting (205-206), based on the first output signal (S1), a need to replenish the first container (23A); and selectively admitting (208) the first fluid from a fluid source (10) into the first container (23A).

C5. The method of any preceding clause, further comprising: operating (203) a third pump (25C) to pump a third fluid from a third container (23C) arranged on the second scale (24B), through a third fluid channel (22C) into the first fluid channel (21) at the junction (26B), or at a further junction (26C) in the first fluid channel (21), to admix the third fluid within the first fluid channel (21), the third fluid being a component of the medical fluid, wherein the third pump (25C) is operated to achieve a second proportion between the second flow rate of the second fluid into the junction (26B) and a third fluid flow rate of the third fluid into the junction (26B) or the further junction (26C).

C6. The method of C5, wherein the third pump (25C) is operated to achieve the second proportion by setting a pumping speed of the third pump (25C) in relation to a pumping speed of the second pump (25B) based on known stroke volumes of the second and third pumps (25B, 25C).

C7. The method of C5 or C6, said method further comprising a verification procedure (600) comprising: determining (601), based on the second output signal (S2), an initial combined value of the second and third flow rates while the second and third pumps (25B, 25C) are operated at a respective initial speed; effecting (602) a first fractional change of the pumping speed of one of the second and thirds pumps (25B, 25C) from its initial speed; determining (603), based on the second output signal (S2), a subsequent combined value of the second and third flow rates resulting from the first fractional change; and evaluating (612) pumping accuracy of said one of the second and third pumps (25B, 25C) based on the initial combined value, the subsequent combined value, and the first fractional change.

C8. The method of C7, wherein said evaluating (612) pumping accuracy comprises: calculating an estimated flow rate value as (Q_{BC,0}-Q_{BC,1})/(1-α1), and comparing the estimated flow rate value to a set value for the second or third flow rate before the first change, wherein Q_{BC,0} is the initial combined value, Q_{BC,1} is the subsequent combined value, and α1 is the first fractional change.

C9. The method of C8, further comprising: performing (613) a dedicated action when a difference between the estimated flow rate value and the set value exceeds a limit value.

C10. The method of any one of C7-C9, wherein the verification procedure (600) comprises: changing (604) the pumping speed of said one of the second and third pumps (25B, 25C) back to the initial speed, determining (605), based on the second output signal (S2), a further subsequent combined value of the second and third flow rates resulting from said changing the pumping speed back to the initial speed, and comparing (606) the initial combined value and the further subsequent combined value.

C11. The method of C10, wherein the verification procedure (600) further comprises: performing (608) a dedicated action when a difference between the initial combined value and the further subsequent combined value exceeds a limit value.

C12. The method of any one of C7-C9, wherein the verification procedure (600) further comprises: determining (605), based on the second output signal (S2), a further initial combined value of the second and third flow rates while the second and third pumps (25B, 25C) are operated at a respective further initial speed; effecting (609) a second fractional change of the pumping speed of said one of the second and third pumps (25B, 25C) from its further initial speed, wherein the second fractional change increases and the first fractional change decreases the pumping speed, or vice versa; and determining (610), based on the second output signal (S2), a further subsequent combined value of the second and third flow rates resulting from the second fractional change, wherein the pumping accuracy of said one of the second and thirds pumps (25B, 25C) is evaluated also based on the further initial combined value, the further subsequent combined value, and the second fractional change.

C13. The method of any one of C7-C12, wherein the verification procedure (600) comprises: changing (604, 609, 611) the pumping speed of said one of the second and third pumps (25B, 25C) for a period of time so as to counteract an increase or decrease in amount of fluid pumped by said one of the second and third pumps (25B, 25C) as a result of said first fractional change.

C14. The method of any preceding clause, further comprising: operating (1101) a valve arrangement (27A; 27′, 27″) to open, intermediate the junction (26B) and an outlet (21B) of the first fluid channel (21), a passage from the first fluid channel (21) to a bypass channel (121); operating (1102) the first pump (25A) to pump the first fluid from the first container (23A) via the first fluid channel (21) into the bypass channel (121); operating (1103) the second pump (25B) to pump the second fluid from the second container (23B) via the second fluid channel (22B), the first junction (26B), and the first fluid channel (21) into the bypass channel (121) to provide a mixture of the first and second fluids in the bypass channel (121); measuring (1104), by a sensor (36), the composition-related parameter of the mixture; adjusting (1105) a pumping speed of at least one of the first and second pumps (25A, 25B) until the sensor (36) measures a target value of the composition-related parameter; and determining (1106), based on first and second output signals (S1, S2) from the first and second scales (24A, 24B), the first proportion as a relation between a first weight change of the first scale (24A) and a second weight change of the second scale (24B) while the sensor (36) measures the target value.

C15. The method of C14, wherein the bypass channel (121) extends to the sensor (36), so that the mixture of the first and second fluids, by said operating (1101) the valve arrangement, said operating (1102) the first pump and said operating (1103) the second pump, is directed through the sensor (36).

C16. The method of any one of C1-C4, further comprising: operating (203) a third pump (25C) to pump a third fluid from a third container (23C) arranged on a third scale (24C), through a third fluid channel (22C) into the first fluid channel (21) at the junction (26B), or at a further junction (26C) in the first fluid channel (21), to admix the third fluid within the first fluid channel (21), the third fluid being a component of the medical fluid, wherein the third pump (25C) is controlled, based on a third output signal (S3) from the third scale (24C), to achieve a second proportion between the second flow rate of the second fluid into the junction (26B) and a third flow rate of the third fluid into the junction (26B) or the further junction (26C).

C17. The method of any of preceding clause, further comprising: requesting (204A) a sample to be taken of the medical fluid downstream of the junction (26B) and composition data for the sample to be input, and, in response to the input of the composition data, adjusting (204B) the first proportion based on the composition data.

C18. The method of any preceding clause, wherein said controlling (204) the first and second pumps comprises: controlling the first flow rate to generate the medical fluid at a flow rate that matches a consumption rate of the medical fluid in an apparatus (100) for renal replacement therapy, which is connected to receive the medical fluid from the first fluid channel (21).

C19. A computer-readable medium comprising computer instructions which, when executed by a processor (41), cause the processor (41) to perform the method of any preceding clause.

C20. A system for generating a medical fluid for use in treatment of blood by renal replacement therapy, said system comprising: a first scale (24A); a first container (23A) arranged on the first scale (24A); a first fluid channel (21) arranged to receive a first fluid from the first container (23A); a first pump (25A) arranged to pump fluid through the first fluid channel (21); a second scale (24B); a second container (23B) arranged on the second scale (24B) and connected, by a second fluid channel (22B), to the first fluid channel (21) at a junction (26B); and a second pump (25B) arranged to pump a second fluid from the second container (23B) through the second fluid channel (22A) into the first fluid channel (21) to admix the second fluid within the first fluid channel (21), the first and second fluids being components of the medical fluid.

C21. The system of C20, further comprising a device (264) which is configured to promote mixing of the second fluid into the first fluid in the first fluid channel (21).

C22. The system of C20 or C21, wherein the first fluid channel (21) further comprises a first end (21A) configured to receive the first fluid from a fluid source (10), the first container (23A) being connected in fluid communication with the first fluid channel (21) between the first end (21A) and the first pump (25A).

C23. The system of any one of C20-C22, wherein the junction (26B) is a three-way connector, and wherein the first fluid channel (21) is at least partly defined by tubing (21′, 21″) attached to first and second ports (261, 262) of the three-way connector and wherein the second fluid channel (22B) is at least partly defined by a tubing (22′) attached to a third port (263) of the three-way connector.

C24. The system of any one of C20-C23, wherein the first and second fluid channels (21, 22B) and the first and second containers (23A, 23B) are combined to form a disposable arrangement (120).

C25. The system of any one of C20-C24, further comprising a bypass channel (121) which is connected to the first fluid channel (21) intermediate the junction (26B) and an outlet (21B) for the medical fluid, and a valve arrangement (27′, 27″) which is operable to selectively direct fluid into one of the first fluid channel (21) or the bypass channel (121).

C26. The system of C25, further comprising a sensor (36) configured to measure a composition-related parameter, wherein the bypass channel (121) extends to the sensor (36).

C27. The system of any one of C20-C26, wherein the second pump (25B) is arranged in the second fluid channel (22B), and the first pump (25A) is arranged in the first fluid channel (21) between the junction (26B) and an outlet (21B) for the medical fluid.

C28. The system of any one of C20-C27, further comprising a control device (40) configured to perform the method of any one of C1-C18.

C29. A disposable arrangement for mounting to an apparatus (100), said disposable arrangement comprising: a first container (23A) configured for mounting on a first scale (24A) of the apparatus (100); a first fluid channel (21) arranged to receive a first fluid from the first container (23A); and a second fluid channel (22B) connected to a junction (26B) on the first fluid channel (21); wherein the first fluid channel (21) defines a first engagement portion (E1) for engagement with a first pump (25A) on the apparatus (100), and wherein the second fluid channel (22B) defines a second engagement portion (E2) for engagement with a second pump (25B) of the apparatus (100) for pumping a second fluid through the second fluid channel (22B) into the first fluid channel (21) to admix the second fluid within the first fluid channel (21), wherein the first and second fluids are components of a medical fluid for use in blood treatment by renal replacement therapy, and wherein the disposable arrangement is operable, when mounted on the apparatus (100), to generate the medical fluid in the first fluid channel (21).

C30. The disposable arrangement of C29, wherein the first fluid channel (21) further comprises an inlet end (21A) configured to receive the first fluid from a fluid source (10), the first container (23A) being connected in fluid communication with the first fluid channel (21) between the inlet end (21A) and the junction (26B).

C31. The disposable arrangement of C29 or C30, wherein the first container (23A) is empty.

C32. The disposable arrangement of any one of C29-C31, wherein the first fluid is water.

C33. The disposable arrangement of any one of C29-C32, further comprising at least one of a second container (23B) in fluid communication with the second fluid channel (22B) or a connector (22B′) on the second fluid channel (22B) for attachment of the second container (23B), the second container (23B) being configured for mounting on a second scale (24B) of the apparatus (100).

C34. The disposable arrangement of C33, wherein the second container (23B) holds the second fluid.

C35. The disposable arrangement of C33 or C34, wherein the second fluid is a liquid concentrate.

C36. The disposable arrangement of any one of C33-C35, further comprising a third fluid channel (22C) connected to the junction (26B), or a further junction (26C) on the first fluid channel (21), wherein the third fluid channel (22C) defines a third engagement portion for engagement with a third pump (25C) of the apparatus (100) for pumping a third fluid through the third fluid channel (22C) into the first fluid channel (21) to admix the third fluid within the first fluid channel (21), wherein the third fluid is a component of the medical fluid.

C37. The disposable arrangement of C36, further comprising at least one of a third container (23C) in fluid communication with the third fluid channel (22C) or a connector on the third fluid channel (22C) for attachment of the third container (23C), the third container (23C) being configured for mounting on the second scale (24B) or a third scale (24C) of the apparatus (100).

C38. The disposable arrangement of C37, wherein the third container (23C) holds the third fluid.

C39. The disposable arrangement of any one of C29-C38, further comprising a sampling port (28) on the first fluid channel (21) downstream of the junction (26B), the sampling port (28) being configured to provide access to the medical fluid in the first fluid channel (21) for sampling.

C40. The disposable arrangement of any one of C29-C39, further comprising a bypass channel (121) in fluid communication with the first fluid channel (21) intermediate the junction (26B) and an outlet end (21B) for the medical fluid, said bypass channel (121) comprising a connector (21C) for releasable attachment to a sensor (36) for measuring a composition-related parameter.

C41. The disposable arrangement of C40, which is configured for mounting on a valve arrangement (105) of the apparatus (100), the valve arrangement (105) being operable to selectively direct fluid in the first fluid channel (21) into one of the outlet (21B) or the bypass channel (121).

1. A method of generating a medical fluid for use in treatment of blood by renal replacement therapy, the method comprising:
operating a first pump to pump a first fluid from a first container arranged on a first scale, through a first fluid channel, the first fluid being a component of the medical fluid;
operating a second pump to pump a second fluid from a second container arranged on a second scale, through a second fluid channel into the first fluid channel at a junction in the first fluid channel, to admix the second fluid within the first fluid channel, the second fluid being a component of the medical fluid; and
controlling the first and second pumps, based on first and second output signals from the first and second scales, to achieve a first proportion between a first flow rate of the first fluid into the junction and a second flow rate of the second fluid into the junction.

2. The method of claim 1, wherein the first and second fluid channels and the first and second containers are combined to form a disposable arrangement, which is replaced during the renal replacement therapy or discarded when the renal replacement therapy is completed.

3. The method of claim 1, wherein the controlling comprises: determining, based on the first and second output signals, a first weight change per unit time for the first scale and a second weight change per unit time for the second scale, and operating the first and second pumps to achieve the first proportion between the first and second weight changes per unit time.

4. The method of any preceding claim 1, further comprising: detecting, based on the first output signal, a need to replenish the first container; and selectively admitting the first fluid from a fluid source into the first container.

5. The method of claim 1, further comprising: operating a third pump to pump a third fluid from a third container arranged on the second scale, through a third fluid channel into the first fluid channel at the junction, or at a further junction in the first fluid channel, to admix the third fluid within the first fluid channel, the third fluid being a component of the medical fluid, wherein the third pump is operated to achieve a second proportion between the second flow rate of the second fluid into the junction and a third fluid flow rate of the third fluid into the junction or the further junction.

6. The method of claim 5, wherein the third pump is operated to achieve the second proportion by setting a pumping speed of the third pump in relation to a pumping speed of the second pump based on known stroke volumes of the second and third pumps.

7. The method of claim 5, the method further comprising a verification procedure comprising:
determining, based on the second output signal, an initial combined value of the second and third flow rates while the second and third pumps are operated at a respective initial speed;
effecting a first fractional change of the pumping speed of one of the second and thirds pumps from its initial speed;
determining, based on the second output signal, a subsequent combined value of the second and third flow rates resulting from the first fractional change; and
evaluating pumping accuracy of one of the second and third pumps based on the initial combined value, the subsequent combined value, and the first fractional change.

8. The method of claim 7, wherein evaluating the pumping accuracy comprises: calculating an estimated flow rate value as (Q_{BC,0}-Q_{BC,1})/(1-α1), and comparing the estimated flow rate value to a set value for the second or third flow rate before the first change, wherein Q_{BC,0} is the initial combined value, Q_{BC,1} is the subsequent combined value, and α1 is the first fractional change.

9. The method of claim 8, further comprising: performing a dedicated action when a difference between the estimated flow rate value and the set value exceeds a limit value.

10. The method of claim 7, wherein the verification procedure comprises: changing the pumping speed of one of the second and third pumps back to the initial speed, determining, based on the second output signal, a further subsequent combined value of the second and third flow rates resulting from changing the pumping speed back to the initial speed, and comparing the initial combined value and the further subsequent combined value.

11. The method of claim 10, wherein the verification procedure further comprises: performing a dedicated action when a difference between the initial combined value and the further subsequent combined value exceeds a limit value.

12. The method of claim 7, wherein the verification procedure further comprises:
determining, based on the second output signal, a further initial combined value of the second and third flow rates while the second and third pumps are operated at a respective further initial speed;
effecting a second fractional change of the pumping speed of one of the second and third pumps from its further initial speed, wherein the second fractional change increases and the first fractional change decreases the pumping speed, or vice versa; and
determining, based on the second output signal, a further subsequent combined value of the second and third flow rates resulting from the second fractional change,
wherein the pumping accuracy of one of the second and thirds pumps is evaluated also based on the further initial combined value, the further subsequent combined value, and the second fractional change.

13. The method of claim 7, wherein the verification procedure comprises: changing the pumping speed of one of the second and third pumps for a period of time so as to counteract an increase or decrease in amount of fluid pumped by one of the second and third pumps as a result of the first fractional change.

14. The method of claim 1, further comprising:
operating a valve arrangement to open, intermediate the junction and an outlet of the first fluid channel, a passage from the first fluid channel to a bypass channel;
operating the first pump to pump the first fluid from the first container via the first fluid channel into the bypass channel;
operating the second pump to pump the second fluid from the second container via the second fluid channel, the first junction, and the first fluid channel into the bypass channel to provide a mixture of the first and second fluids in the bypass channel;
measuring, by a sensor, the composition-related parameter of the mixture;
adjusting a pumping speed of at least one of the first and second pumps until the sensor measures a target value of the composition-related parameter; and
determining, based on the first and second output signals from the first and second scales, the first proportion as a relation between a first weight change of the first scale and a second weight change of the second scale while the sensor measures the target value.

15. The method of claim 14, wherein the bypass channel extends to the sensor, so that the mixture of the first and second fluids, by operating the valve arrangement, the operating the first pump and the operating the second pump, is directed through the sensor.

16. The method of claim 1, further comprising: operating a third pump to pump a third fluid from a third container arranged on a third scale, through a third fluid channel into the first fluid channel at the junction, or at a further junction in the first fluid channel, to admix the third fluid within the first fluid channel, the third fluid being a component of the medical fluid, wherein the third pump is controlled, based on a third output signal from the third scale, to achieve a second proportion between the second flow rate of the second fluid into the junction and a third flow rate of the third fluid into the junction or the further junction.

17. The method of claim 1, further comprising: requesting a sample to be taken of the medical fluid downstream of the junction and a composition data for the sample to be input, and, in response to the input of the composition data, adjusting the first proportion based on the composition data.

18. The method of claim 1, wherein said controlling the first and second pumps comprises: controlling the first flow rate to generate the medical fluid at a flow rate that matches a consumption rate of the medical fluid in an apparatus for renal replacement therapy, which is connected to receive the medical fluid from the first fluid channel.

19. A computer-readable medium comprising computer instructions which, when executed by a processor, cause the processor to perform the method of claim 1.

20. A system for generating a medical fluid for use in treatment of blood by renal replacement therapy, the system comprising:
a first scale;
a first container arranged on the first scale;
a first fluid channel arranged to receive a first fluid from the first container;
a first pump arranged to pump fluid through the first fluid channel;
a second scale;
a second container arranged on the second scale and connected, by a second fluid channel, to the first fluid channel at a junction; and
a second pump arranged to pump a second fluid from the second container through the second fluid channel into the first fluid channel to admix the second fluid within the first fluid channel, the first and second fluids being components of the medical fluid.

21. The system of claim 20, further comprising a device which is configured to promote mixing of the second fluid into the first fluid in the first fluid channel.

22. The system of claim 20, wherein the first fluid channel further comprises a first end configured to receive the first fluid from a fluid source, the first container being connected in fluid communication with the first fluid channel between the first end and the first pump.

23. The system of claim 20, wherein the junction is a three-way connector, and wherein the first fluid channel is at least partly defined by tubing attached to first and second ports of the three-way connector and wherein the second fluid channel is at least partly defined by a tubing attached to a third port of the three-way connector.

24. The system of claim 20, wherein the first and second fluid channels and the first and second containers are combined to form a disposable arrangement.

25. The system of claim 20, further comprising a bypass channel which is connected to the first fluid channel intermediate the junction and an outlet for the medical fluid, and a valve arrangement which is operable to selectively direct fluid into one of the outlet or the bypass channel.

26. The system of claim 25, further comprising a sensor configured to measure a composition-related parameter, wherein the bypass channel extends to the sensor.

27. The system of claim 20, wherein the second pump is arranged in the second fluid channel, and the first pump is arranged in the first fluid channel intermediate the junction and an outlet for the medical fluid.

28. The system of claim 20, further comprising a control device configured to perform the method of claim 1.

29-41. (canceled)
